(19) United States
(12) Patent Application Publication
(10) Pub. No.: US 2022/0395555 A1
(43) Pub. Date: Dec. 15, 2022

(54) Derivatives of antibiotics

(71) Applicants: Boris Farber, Brooklyn, NY (US); Sof'ya Farber, Brooklyn, NY (US); ARTUR VIKTOROVICH MARTYNOV, Kharkov (UA)

(72) Inventors: Boris Farber, Brooklyn, NY (US); Sof'ya Farber, Brooklyn, NY (US); ARTUR VIKTOROVICH MARTYNOV, Kharkov (UA)

(21) Appl. No.: 17/890,503

(22) Filed: Aug. 18, 2022

Related U.S. Application Data
(63) Continuation-in-part of application No. 16/771,762, filed on Jun. 11, 2020.

(51) Int. Cl. A61K 38/12; A61K 31/7036; A61K 31/65; A61K 31/40; A61K 31/7048

(52) U.S. Cl. CPC A61K 38/12; A61K 31/7036; A61K 31/65; A61K 31/40; A61K 31/7048

(57) ABSTRACT

Field of application: The invention relates to chemistry, pharmacy and cosmetology, allows to synthesize of supramolecular structure on base antibiotics derivatives for use in pharmacy, cosmetology and pharmacy.

The essence of the invention: a new derivatives of antibiotics based on supramolecular structures and a method for their preparation, characterized in that the supramolecular structures are obtained by combinatorial synthesis of an antibiotic from one source molecule with two or more groups available in the reaction for covalent modification, at least with two different modifiers simultaneously, this creates a mixture of modified derivatives of the original molecule, with a maximum variety of derivatives with forming new supramolecular structure, and as biologically active substances to create pharmaceutical compositions use such supramolecular structure without separation into individual components, and in the reaction a combinatorial mixture of modified derivatives of the original molecule is formed antibiotic, the maximum number of combinations.

Technical result: modified combinatorial derivatives of antibiotics with antimicrobial and antifungal activity against multiresistant and pan drug resistance strains of microorganisms and fungi. Means have a wide spectrum of action, and the supramolecular and combinatorial structure of their tens and hundreds of derivatives eliminates the resistance of microorganisms.

### TECHNICAL FIELD

The invention relates to chemistry, pharmacy and cosmetology. It allows you to synthesize new derivatives of antibiotics for use in pharmacy, cosmetology and pharmacy.

### STATE OF THE ART

Combinatorial chemistry, the methodology of organic chemical synthesis, is a set of techniques and methods for combining diverse source chemicals to produce as many arrays of chemical products as possible. This is done by conducting tens, hundreds, and sometimes thousands of parallel chemical transformations with the formation of a huge number of final products. Combinatorial chemistry solves problems rarely arising in classical chemical synthesis, specifically to quickly synthesize many substances, usually complex in structure and fairly pure structures.

The development of new economical and speedy technologies for parallel synthesis and parallel purification of substances is achieved in a variety of ways. Instead of the standard liquid-phase synthesis (one substance in one vessel at a time), many syntheses (for example, in a plastic plate with many cells, where the substances are introduced by multichannel pipettes). Instead of boiling under reflux, they use the heating of many sealed capsules (in a cellular thermostat or microwave oven). To filter multiple substances “filter vessels” usually used (for example, dies with a porous bottom).

Evaporation is carried out by vacuum freezing of the solvent from centrifuged dies (it prevents foaming). For purification methods of parallel chromatography used, combining in blocks of many chromatographic columns. In methods of liquid-phase combinatorial chemistry, they try to use only those reactions that result in high yields and require minimal effort to purify the substances. In order to achieve a wider variety of products, conventional two-component reactions are replaced by multicomponent ones.

A powerful technology of combinatorial chemistry is solid-phase synthesis. This process consists of carrying out reactions on a modified polymer substrate. Solid-phase synthesis is a complex molecule (for example, a polypeptide of the desired sequence or a complex heterocyclic compound) is immobilized (“built up”) on the surface of the polymer during the sequence of reactions, and then, at the final stage, it is cleaved from the solid substrate as result of any chemical transformations. Therefore, reactions can be carried out with a large excess of reagent, washing the latter from the polymer with the target substance and reducing the synthesis to the principle of “tea bag” (porous bags with polymer granules are successively placed in glasses with reagents). A new technology is the replacement of solid polymers with perfluorinated liquids (not miscible with water and standard solvents).

For immobilization (transfer of the substance to the perfluorinated phase), an extended perfluoroalkyl moiety is attached to the molecule of the starting reagent. This allows forsynthesis in emulsions, followed by separation of the liquid phases. The combined method of combinatorial chemistry is the use of solid-phase reagents (oxidizing agent, acid, base are immobilized on a polymer). Excess solid reagent is added to the solutions of substances, and then separated by filtration.

Another useful technique is use scavengers—a modified polymer is introduced into the solution, which selectively removes excess reagent from the reaction mixture, taken in excess. Programmed industrial robots are increasingly being used, performing a sequence of routine uniform procedures for the isolation and purification of substances (automatic synthesizers). The effectiveness of combinatorial chemistry has been proven by numerous examples of discovered new drugs and catalysts [¹,²]. 1 Handbook of combinatorial chemistry: drugs, catalysts, materials. Wcinhcim, 2002. Vol. 1-22 Combinatorial chemistry on solid supports. B , 2007

Our proposed synthesis scheme for combinatorial derivatives based on one polyfunctional source molecule (for example, polymyxin) by reaction with two or more source modifiers without subsequent separation and isolation of each individual derivative is unique and showed an increase in biological activity from two to 300 times for different source molecules including: polymyxin, gentamicin, streptomycin, individual oligomeric RNA and DNA, polysaccharides, proteins, quercetin and many other substances.

An important innovation in this approach is the correct calculation of the molar ratio of the number of reagents, both the initial polyfunctional compound (in this case, antibiotics) and modifiers. With the correct ratio of components, the maximum possible combination of derivatives is formed. This mixture is not a classical solution or a mixture after synthesis, rather in aqueous solutions it forms supramolecular structures with each other in arbitrary positions and behaves like the original antibiotic, but with a more pronounced biological activity and prolonged action.

The formation of supramolecular structures can be traced by the absence of separation of the band of the combinatorial derivative at the chromatographic peak. Especially any changes in the separation conditions could not lead to separation of the mixture. At the same time, in the H1 NMR spectrum, there was clear chaos from the absorption bands of hydrogen of both methyl groups of the acetic acid residue and ethyl groups of the succinic acid residue and un substituted phenyl hydroxides.

### Antibiotics

Antibiotics (from the Greek. And—prefix, meaning counteraction, and bios-life), substances synthesized by microorganisms, and products of chemical modification of these substances that selectively inhibit the growth of pathogenic microorganisms, lower fungi, as well as some viruses and cancer cells. There are more than 6 thousand naturally occurring antibiotics have been described, but only about 50 of them are widely used. When determining the effectiveness of antibiotics, consider their antimicrobial activity, the degree of penetration into the lesion foci, the possibility of creating therapeutic concentrations in the patient's tissues and fluids and the duration of their maintenance, the rate of development of resistance by microorganisms during treatment, preservation of antibiotics action under various conditions are also taken into account.

Most antibiotics are obtained in industry by microbiological synthesis—in fermenters on special nutrient media. The antibiotics synthesized by microorganisms are recovered and chemically purified using various methods. The main producers of antibiotics are soil microorganisms—radiant mushrooms (actinomycetes), mold fungi and bacteria. Molecules of natural antibiotics do not always have satisfactory chemotherapeutic and pharmacological properties. In addition, resistant forms of microorganisms with the ability to destroy antibiotics, mainly by exposure to them with their enzymes, are widely used.

Therefore, the main direction of creating new antibiotics is chemical and microbiological modifications of natural antibiotics and the production of semi-synthetic antibiotics. About 100 thousand semi-synthetic antibiotics have been described, but only a few have properties that are valuable to medicine. A number of natural antibiotics, especially benzylpenicillin, cephalosporin, sifamycin, are mainly used to obtain semi-synthetic derivatives. For a number of antibiotics, methods of complete chemical synthesis have been developed, which, however, are complex and not economically justified. Only chloramphenicol and cycloserine are synthetically produced.

Antibiotics belong to the most diverse classes of chemical compounds—amino sugars, anthraquinones, glycosides, lactones, phenazines, piperazines, pyridines, quinones, terpenoids and others. Of greatest importance are lactam antibiotics (penicillins and cephalosporins), macrolide antibiotics, ansamycins, aminoglycoside antibiotics, tetracyclines, peptide antibiotics, anthraikyline.

The following antibiotic groups are distinguished by the molecular mechanism of action: 1) inhibitors of the synthesis of the cell wall of microorganisms (penicillins, cycloserine, and others); 2) inhibitors of membrane functions and having detergent properties (polyenes, novobiocin); 3) inhibitors of protein synthesis and ribosome functions (tetracyclines, macrolide antibiotics); 4) inhibitors of RNA metabolism (for example, actinomycin, anthracyclines) and DNA (mitomycin C, streptonigrin).

Knowledge of the mechanism of action of the antibiotic allows us to judge not only about the direction of the chemotherapeutic effect (the “target” of the antibiotic), but also about the degree of its specificity. So, lactam antibiotics act on peptidoglycan, a supporting polymer of the bacterial cell wall, which is absent in animals and humans, which determines the high selectivity of these antibiotics.

The following antibiotics are distinguished by their focus (spectrum) of action: 1) active against gram-positive microorganisms—macrolide antibiotics, lincomycin, fusidine and others; 2) a wide spectrum of action, active against both gram-positive and gram-negative microorganisms, tetracyclines, aminoglycosides and others.; 3) anti-tuberculosis-streptomycin, kanamycin, rifampicin, cycloserine and others; 4) antifungal—mainly polyenes, for example nystatin, levorin, griseofulvin; all of them act on the cytoplasmic membrane of pathogenic fungi; effective for mycoses of various etiologies; 5) active against protozoa-trichomycin, paromomycin; 6) antitumor—actinomycin, anthracyclines, bleomycin; inhibit the synthesis of nucleic acids; as a rule, they are used in combination with other drugs (including hormones) along with radiation therapy and surgical treatment. A number of antibiotics, in particular rifamycin derivatives, have antiviral activity, but are not used in the treatment of diseases of viral etiology.

With prolonged use, some antibiotics may have toxic effects on the center of nervous system, auditory nerve, etc., suppress the body's immunobiological reactions, cause allergic reactions. By the severity of side effects, antibiotics do not surpass other groups of drugs. Antibiotics are used to treat human and animal diseases, plant protection, in animal husbandry to improve the growth and development of young animals (antibiotics used as an additives to feed), and in the food industry for canning products.

However, their uncontrolled use can lead to undesirable consequences, primarily to the spread of antibiotic-resistant pathogens of extrachromosomal nature, which cause severe human diseases, as well as to allergic reactions due to residual amounts of antibiotics in food products. The legislation of several countries prohibits or restricts the use of the same antibiotics in human medicine, animal husbandry and the food industry. Some antibiotics are widely used in biochemical and molecular biological studies as specific inhibitors of certain metabolic processes in the cells of living organisms.

### Definitions

Antimicrobial activity. The term “antimicrobial activity” is defined here as the ability to destroy microbial cells or inhibit their growth. It is understood that in the context of the present invention, the term “antimicrobial” means the presence of a bactericidal and/or bacteriostatic and/or fungicidal and/or fungistatic and/or virucidal effect, where the term “bactericidal” is to be understood as capable of killing bacterial cells. The term “bacteriostatic” should be understood as capable of inhibiting bacterial growth, that is, inhibiting the growth of bacterial cells. The term “fungicidal” should be understood as capable of killing fungal cells. The term “fungistatic” should be understood as capable of inhibiting fungal growth, that is, inhibiting the growth of fungal cells.

The term “virucidal” should be understood as capable of inactivating the virus. The term “microbial cells” refers to bacterial or fungal cells (including yeast).

It is understood that in the context of the present invention, the term “inhibition of microbial cell growth” means that these cells are not in a state of growth, that is, that they are not capable of reproduction.

In a preferred embodiment, the term “antimicrobial activity” is defined as bactericidal and/or bacteriostatic activity. More preferably, “antimicrobial activity” is defined as bactericidal and/or bacteriostatic activity against Escherichia, preferably Escherichia coli.

For the objectives of the present invention, antimicrobial activity can be determined by the method described by Lehrer et al., Journal of Immunological Methods, Vol. 137 (2) pp. 167-174 (1991). Alternatively, antimicrobial activity can be determined in accordance with the guidelines of the National Committee for Clinical Laboratory Standards (NCCLS) from CLSI (Clinical and Laboratory Standards Institute), formerly known as the National Committee for Clinical and Laboratory Standards clinical and laboratory standards).

Antimicrobial supramolecular combinatorial antibiotics (ASCA) may be able to reduce the number of viable Escherichia coli cells (DSM 1576) to 1/100 after 8 hours (preferably after 4 hours, more preferably after 2 hours, most preferably after 1 hour and, in particular, after 30 minutes) incubation at 37° C. in an appropriate microbial growth substrate at a concentration of SKA having antimicrobial activity, 500 μg/ml, preferably 250 μg/ml, more preferably 100/ml, even more preferably 50 μg/ml, most preferably 25 μg/ml, and in particular 10 μg/ml.

ASCA with antimicrobial activity may also be able to inhibit the growth of Escherichia coli (DSM 1576) for 8 hours at 37° C. in an appropriate microbial growth substrate when added at a concentration of 500 μg/ml, preferably when added at a concentration of 250 μg/ml, more preferably when they are added at a concentration of 100 μg/ml, even more preferably when they are added at a concentration of 50 μg/ml, most preferably when they are added at a concentration of 10 μg/ml and, in particular, when added at a concentration of 5 mcg/ml.

Known antimicrobial combinatorial libraries of synthetic peptides [3]. We are talking about various synthetic peptides and their amino acid sequence. Substances showed activity in a relatively wide range of microorganisms. The authors showed that these derivatives are active in vitro. The prototype has several disadvantages: patented peptides are not active against multiresistant and pan drug resistant strains and belong to only one group of antibiotics-peptide antibiotics, are used exclusively individually, and not in the form of supramolecular mixtures, are specially divided into separate peptides for use. U.S. Patent US5602097A, appl US08305768

Also, the peptides from the prototype have such disadvantages as sensitivity to digestive enzymes of the intestine and tissues, a narrow spectrum of biological activity, and the impossibility of oral use.

Our proposed combinatorial supramolecular antibiotics are insensitive to digestive enzymes, have a wide spectrum of antimicrobial activity, can be taken orally, and are effective in the treatment of infectious diseases caused by multiresistant strains of microorganisms.

### DISCLOSURE OF INVENTION

A basis of the invention is task to develop new derivatives of antibiotics: polymyxin, amikacin, gentamicin, tetracycline, lincosamine, gramicidin, nystatin, erythromycin, nystatin, which are obtained by the reaction of acylation or/and alkylation of the original antibiotic simultaneously with at least two different modifiers, wherein synthesized individual derivatives self-organize into a supramolecular structure, while the molar ratio of antibiotic to modifiers in the reaction of combinatorial synthesis is calculated by the formulas:

k=n(d+1)ⁿ⁻¹ (1)

m=(d+1)ⁿ−1 (2)

wherein:

n=a number of groups available for substitution in the antibiotic molecule;

m=a number of moles of the antibiotic and the number of different derivatives after synthesis;

k=a number of moles of each of d modifiers in the combinatorial synthesis reaction to obtain the maximum number of different derivatives;

d=a number of modifiers in combinatorial reaction;

and this synthesized supramolecular structure used for creating pharmaceutical compositions.

The modifiers are acylating agents of the group of anhydrides of organic mono—and polycarboxylic acids, halides of carboxylic acids, alkylating agents halogen derivatives of hydrocarbons, acylating agent—mono- or polycarboxylic acid anhydride or carboxylic acid halide.

### BRIEF DESCRIPTION OF THE DRAWINGS

FIG. 1. Scheme of combinatorial synthesis of polymyxin derivatives with the formation of supramolecular combinatorial derivative (IVa-d): polymyxin reacts with two modifying agents—succinic anhydride and acetic anhydride in the calculated proportions. In this case, a supramolecular structure of 380 polymyxin derivatives is formed.

FIG. 2. Scheme of combinatorial synthesis of tetracycline derivatives with the formation of supramolecular combinatorial derivative (VIIa-d): tetracycline reacts with two modifying agents—succinic anhydride and acetic anhydride in the calculated proportions. In this case, a supramolecular structure of 92 tetracycline derivatives is formed.

FIG. 3. TLC of tetracycline derivatives, water: AcCN=1:1, manifestations of a UV lamp (190-300 nm).

FIG. 4. Scheme of combinatorial synthesis of the supramolecular combinatorial derivative of gentamicin (IXa-d): gentamicin (base) reacts with two modifying agents—succinic anhydride and acetic anhydride in the calculated proportions. In this case, a supramolecular structure of 764 gentamicin derivatives is formed.

FIG. 5. TLC of gentamicin derivatives, water: AcCN=1:1, manifestation of a UV lamp (190-300 nm) after treatment with a solution of sulfuric acid.

### Pharmaceutical Compositions

Various methods of administering supramolecular combinatorial antibiotic derivatives (ASCA) can be used. The ASCA composition can be given orally or can be administered by intravascular, subcutaneous, intraperitoneal injection, in the form of an aerosol, by ocular route of administration, into the bladder, topically, and so on. For example, inhalation methods are well known in the art. The dose of the therapeutic composition will vary widely depending on the particular antimicrobial ASCA administered, the nature of the disease, frequency of administration, route of administration, clearance of the agent used from the host, and the like.

The initial dose may be higher with subsequent lower maintenance doses. The dose can be administered once a week or once every two weeks, or divided into smaller doses and administered once or several times a day, twice a week, and so on to maintain an effective dose level. In many cases, a higher dose will be needed for oral administration than for intravenous administration. The compounds of this invention may be included in a variety of compositions for therapeutic administration.

More specifically, the compounds of the present invention can be incorporated into pharmaceutical compositions in combination with suitable pharmaceutically acceptable carriers or diluents, and can be incorporated into preparations in solid, semi-solid, liquid or gaseous forms, such as capsules, powders, granules, ointments, creams, foams, solutions, suppositories, injections, forms for inhalation use, gels, microspheres, lotions and aerosols. As such, the administration of the compounds can be carried out in various ways, including oral, buccal, rectal, parenteral, intraperitoneal, intradermal, transdermal, intratracheal administration and so on.

The ASCA according to the invention can be distributed systemically after administration or can be localized using an implant or other composition that holds the active dose at the site of implantation. The compounds of the present invention can be administered alone, in combination with each other, or they can be used in combination with other known compounds (eg, perforin, anti-inflammatory agents, and so on). In pharmaceutical dosage forms, the compounds may be administered in the form of their pharmaceutically acceptable salts. The following methods and excipients are given as examples only and are in no way limiting.

For preparations for oral administration, the compounds can be used alone or in combination with suitable additives for the manufacture of tablets, powders, granules or capsules, for example, with conventional additives such as lactose, mannitol, corn starch or potato starch; with binding agents, such as crystalline cellulose, cellulose derivatives, gum arabic, corn starch or gelatins; with disintegrants, such as corn starch, potato starch or sodium carboxymethyl cellulose; with lubricants such as talc or magnesium stearate; and, if desired, with diluents, buffers, wetting agents, preservatives and flavoring agents.

The compounds can be incorporated into injectable compositions by dissolving, suspending or emulsifying them in an aqueous or non-aqueous solvent such as vegetable or other similar oils, synthetic aliphatic acid glycerides, esters of higher aliphatic acids or propylene glycol; and, if desired, with conventional additives, such as solubilizers, isotonic agents, suspending agents, emulsifiers, stabilizers and preservatives.

The compounds may be used in an aerosol composition for inhalation administration. The compounds of the present invention can be incorporated into suitable pressure propellants such as dichlorodifluoromethane, propane, nitrogen and the like. In addition, the compounds can be incorporated into suppositories by mixing with a variety of bases, such as emulsifying bases or water-soluble bases.

The compounds of the present invention can be administered rectally using a suppository. A suppository may contain excipients, such as cocoa butter, carboax, and polyethylene glycols, which melt at body temperature but are solid at room temperature. Standard dosage forms for oral or rectal administration, such as syrups, elixirs and suspensions, where each unit dose, for example, a teaspoon, tablespoon, tablet or suppository, may contain a predetermined amount of a composition containing one or more compounds of the present invention.

Similarly, unit dosage forms for injection or intravenous administration may contain the compound of the present invention in a composition in the form of a solution in sterile water, normal saline, or another pharmaceutically acceptable carrier. Implants for the sustained release of compositions are well known in the art. Implants are made in the form of microspheres, plates, and so on with biodegradable or non-biodegradable polymers. For example, lactic and/or glycolic acid polymers form a degradable polymer that is well tolerated by the host.

An implant containing the antimicrobial combinatorial antibiotics of the invention is positioned close to the site of infection, so that the local concentration of the active agent is increased compared to other areas of the body. As used herein, the term “unit dosage form” refers to physically discrete units suitable for use as single doses for subjects of humans and animals, each unit containing a predetermined number of compounds of the present invention, which, according to calculations, is sufficient to provide the desired effect together with a pharmaceutically acceptable diluent, carrier or excipient.

The descriptions of the unit dosage forms of the present invention depend on the particular compound used, and the effect to be achieved, and the pharmacodynamics of the compound used in the host. Pharmaceutically acceptable excipients, such as excipients, adjuvants, carriers or diluents, are generally available. In addition, pharmaceutically acceptable excipients are generally available, such as pH adjusting agents and buffering agents, tonicity agents, stabilizers, wetting agents and the like.

Typical doses for systemic administration range from 0.1 pg to 100 milligrams per kg of subject body weight per administration. A typical dose may be a single tablet for administration from two to six times a day, or one capsule or sustained release tablet for administration once a day with a proportionally higher content of the active ingredient. The effect of prolonged release may be due to the materials of which the capsule is made, dissolving at different pH values, capsules providing a slow release under the influence of osmotic pressure or any other known controlled release method.

Those skilled in the art will appreciate that dose levels may vary depending on the particular compound, the severity of symptoms, and the subject's predisposition to side effects. Some of the specific compounds are more potent than others. Preferred doses of this compound can be readily determined by those skilled in the art in a variety of ways. A preferred method is to measure the physiological activity of the compound. One of the methods of interest is the use of liposomes as a vehicle for delivery.

Liposomes fuse with the cells of the target region and provide delivery of liposome contents to the cells. The contact of the liposomes with the cells is maintained for a time sufficient for fusion using various methods of maintaining contact, such as isolation, binding agents and the like. In one aspect of the invention, liposomes are designed to produce an aerosol for pulmonary administration. Liposomes can be made with purified proteins or peptides that mediate membrane fusion, such as Sendai virus or influenza virus and so on. Lipids can be any useful combination of known liposome forming lipids, including cationic or zwitterionic lipids, such as phosphatidylcholine.

The remaining lipids will usually be neutral or acidic lipids, such as cholesterol, phosphatidylserine, phosphatidylglycerol and the like. To obtain liposomes, the method described by Kato et al. (1991) J. Biol. Chem. 266: 3361. Briefly, lipids and a composition for incorporation into liposomes containing combinatorial supramolecular antibiotics are mixed in a suitable aqueous medium, suitably in a salt medium, where the total solids content will be in the range of about 110 wt. %.

After vigorous stirring for short periods of approximately 5-60 seconds, the tube is placed in a warm water bath at approximately 25-40° C. and this cycle is repeated approximately 5-10 times. The composition is then sonicated for a suitable period of time, typically approximately 1-10 seconds, and optionally further mixed with a vortex mixer. Then the volume is increased by adding an aqueous medium, usually increasing the volume by about 1-2 times, followed by agitation and cooling. The method allows to include supramolecular structures with high total molecular weight in liposomes.

### Compositions with Other Active Agents

For use in the methods under consideration, the ASCA according to the invention can be included in compositions with other pharmaceutically active agents, in particular other antimicrobial agents, immunomodulators, antiviral agents, antiviral substances. Other agents of interest include a wide range of unmodified antibiotics known in the art. Antibiotic classes include penicillins, for example penicillin G, penicillin V, methicillin, oxacillin, carbenicillin, nafcillin, ampicillin and so on; penicillins in combination with betalactamase inhibitors; cephalosporins, for example cefaclor, cefazolin, cefuroxime, moxalactam, etc. etc.

Antifungal agents are also useful, including polyenes, such as amphotericin B, nystatin, flucosin; and azoles, such as miconazole, ketoconazole, itraconazole, and fluconazole. Anti-TB drugs include isoniazid, ethambutol, streptomycin and rifampin. Other agents of interest in terms of creating new compositions include a wide range of mononucleotide derivatives and other RNA polymerase inhibitors known in the art.

Classes of antiviral agents include interferons, lamivudine, ribavirin, etc. Other groups of antiviral agents include adefovir, vbacavir, didanosine, emtricitabine, lamivudine, stavudine, tenofovir, efavirenz, nevirapine, indinavir, lopinavir and ritonavir, nelfinavir, ritonavir, sakinavir, daclatasvir, Sovof. Cytokines, such as interferon gamma, tumor necrosis factor alpha, interleukin 12, and so on, may also be included in the antimicrobial SCA composition of the invention. The present invention is further described by the following examples, which should not be construed as limiting the scope of the invention.

### Example 1. Obtaining a Supramolecular Combinatorial Mixture of Polymyxin (PPC)

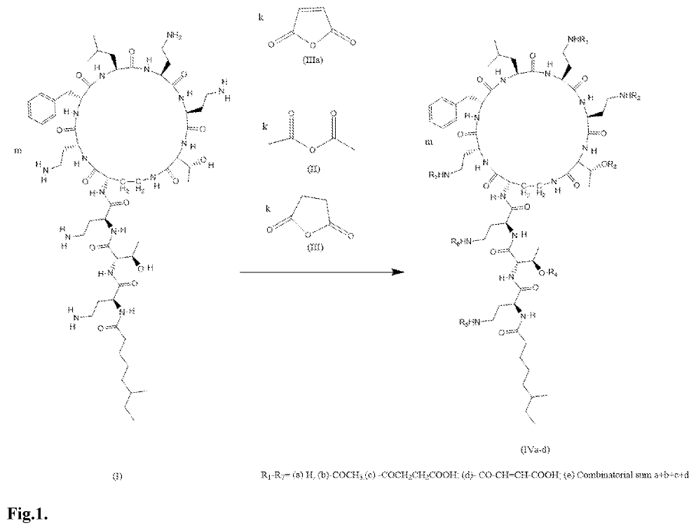

164 μM polymyxin B (I) is dissolved in 10 ml of dioxane (CAS N 1404-26-8, Mr=1203.499 g/mol, n=7) (I), 287 μM succinic anhydride (III), 287 μM acetic anhydride (II), 287 μM maleic anhydride are added, the solution is stirred and heated under reflux for 10 minutes. The solution was poured into ampoules and lyophilized to remove solvent and acetic acid. The combinatorial mixture (IVa-d) is used to obtain pharmaceutical compositions, study the structure, determine the biological activity. FIG. 1 shows a synthesis scheme for combinatorial derivatives of polymyxin.

FIG. 1. One initial polymyxin molecule contains 7 peptide residues of amino groups and hydroxyl groups available for

Calculations of the number of moles of modifiers are carried out according to the combinatorics formulas:

(1)k=n(d+1)ⁿ⁻¹;(2)m=(d+1)ⁿ−1,

wherein

m=a number of different derivatives of molecules in the combinatorial mixture and the number of moles of polymyxin for the reaction; n- is the number of amino groups and hydroxyl groups available for modification in the structure of polymyxin (n=7);

k=a number of moles of each modifier;

d=a number of modifiers (3 in our case—succinic anhydride, acetic anhydride and maleic anhydride).

Therefore, having only one initial polymyxin molecule and two modifiers after combinatorial synthesis, we obtain 16 384 combinatorial derivatives with different degrees of substitution, different positions of substituents and different permutations of the modifier residues, not just as a mixture, but as a difficult to separate modification (n=7), supramolecular mixture.

Due to the presence in both derivatives of both substituted and non-substituted hydroxyl and amino groups, supramolecular structures are formed through both hydrogen and ionic bonds. Modifiers-succinic anhydride or acetic anhydride can be entered both simultaneously and sequentially—or first inject succinic anhydride, warm the mixture under reflux for 10 minutes, and then add acetic anhydride and also warm the mixture for another 10 minutes. Similarly, in this reaction, succinic anhydride, maleic anhydride, aconitic anhydride, glutaric, phthalic anhydride and acetic anhydride, formic acid ethyl ester, monochloroacetic acid, propiolactone, ethylene oxide, and other low-chloro chlorides can be used as one of the d modifiers)

For the HPLC, a Milichrom A-02 microcolumn chromatograph in a gradient of acetonitrile (5-100%)/0.1 M perchloric acid+0.5 M lithium perchlorate was used. The combinatorial derivative in the chromatogram gave one clear broadened peak and was not separated into components, although the retention time differed from both the original polymyxin and its completely substituted derivatives. This testified to the fact that complex supramolecular structures were formed between different combinatorial derivatives (in our case, 16 383), which were not separated chromatographically. This combinatorial derivative (CPP) behaves similarly and when separated in a thin layer (acetonitrile: water) gives only one band, which does not coincide with any of the obtained derivatives. An attempt to use two-dimensional TLC in different conditions also did not allow us to separate the combinatorial derivative. This is a characteristic feature of the supramolecular structure in the combinatorial derivative (IVa-d).

### Example 2. Obtaining a Supramolecular Combinatorial Mixture of Tetracycline (CBT)

243 μM tetracycline (VI) is dissolved in 10 ml of dioxane (CAS N 60-54-8, Mr=444.44 g/mol, n=5) (I), 405 μM succinic anhydride (III) and 405 μM acetic anhydride (II) are added, the solution is stirred and heated under reflux for 10 minutes, 1200 μM TRIS is added to the solution, stirred until dissolved. The solution was poured into ampoules and lyophilized to remove solvent and acetic acid. The combinatorial mixture (VIIa-d) is used to obtain pharmaceutical compositions, study the structure, determine the biological activity (CBT). FIG. 1 shows a synthesis scheme for combinatorial derivatives of tetracycline.

In this reaction, instead of tetracycline, oxytetracycline or any other tetracycline derivative with hydroxyl groups available for modification can be used, as well as any other antibiotic with two or more groups available for modification: aminoglycoside antibiotics, polyene antibiotics, tetracycline, macrolide antibiotics, lincosamine, gramicidin, glycopeptide antibiotics. Instead of modifiers of carboxylic acid anhydrides, halides of carboxylic and polycarboxylic acids, such as succinic, maleic, fumaric, lactic, propionic, other halogen derivatives, such as chloromethane, bromoethane, chloropropane, cyclic alkylating compounds, such as oxirane, and propoxy, can be used.

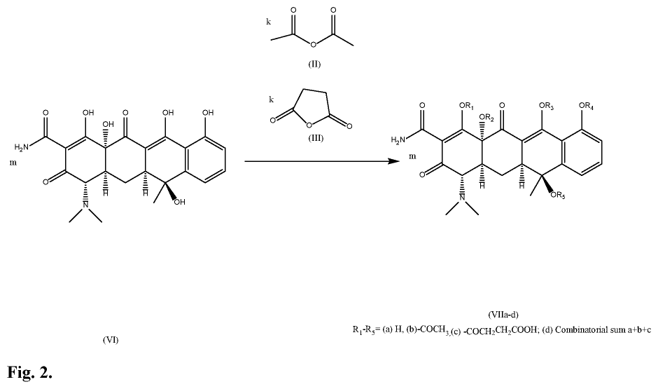

FIG. 2.

One source molecule of tetracycline (I) contains 5 hydroxyl groups available for modification (n=5).

Calculations of the number of moles of modifiers are carried out according to the combinatorics formulas:

(1)k=n(d+1)ⁿ⁻¹;(2)m=(d+1)ⁿ−1

, wherein

m=a number of different derivatives of molecules in the combinatorial mixture and the number of moles of tetracycline for the reaction; n- is the number of amino groups and hydroxyl groups available for modification in the structure of polymyxin (n=5);

k=a number of moles of each modifier;

d−a number of modifiers (2 in our case—succinic anhydride, acetic anhydride).

Thus, having only one initial tetracycline molecule and two modifiers after combinatorial synthesis, we obtain 242 combinatorial derivatives with different degrees of substitution, different positions of substituents and different permutations of the modifier residues, not just as a mixture, but as a difficult to separate supramolecular mixture.

Due to the presence of both substituted and non-substituted hydroxyl groups in various derivatives, supramolecular structures are formed through both hydrogen and ionic bonds. Modifiers—succinic anhydride or acetic anhydride can be entered both simultaneously and sequentially—or first introduce succinic anhydride, warm the mixture under reflux, and then add acetic anhydride and re-warm the mixture. Similarly, in this reaction, maleic anhydride, aconitic anhydride, glutaric, phthalic anhydride and acetic anhydride, formic acid ethyl ester, monochloroacetic acid, propiolactone, ethylene oxide, and other low-chloro chlorides can be used as one of the modifiers instead of succinic anhydride.)

For the HPLC, a Milichrom A-02 microcolumn chromatograph in a gradient of acetonitrile (5-100%) / 0.1 M perchloric acid+0.5 M lithium perchlorate was used. The combinatorial derivative in the chromatogram gave one clear broadened peak and was not separated into components, although the retention time differed from both the original polymyxin and its completely substituted derivatives. This indicated that complex supramolecular structures were formed between different combinatorial derivatives (in our case, 242), which were not separated chromatographically. This combinatorial derivative (PMM) behaves similarly when separated in a thin layer (acetonitrile: water, UV detection) and gives only one band, which does not coincide with any of the obtained derivatives.

NMR C13: C: 199.4; 197.6; 169.5; 149.9; 156.2; 93.4; 83.1; CH: 76.7; C: 108.6; 116.4; 143.5; 106.2; CH: 117.1; 120.7; 128.1; 27.4; 38.6; CH2: 14.7; C: 147.5; 171.1; 173.1; 174.7; 172.0; CH3: 44.6; 24.0; CH2: 28.8; 29.8; 29.1

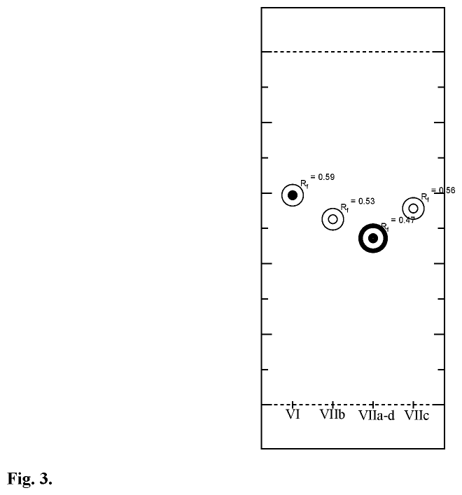

FIG. 3.

As can be seen from TLC, the Combinatorial mixture (lane VIIa-d) is less mobile and has Rf=0.47, while the initial unmodified tetracycline (VI) is the lightest and Rf=0.59. Fully acylated tetracycline (VIIb) and succinyl tetracycline (VIIC) are intermediate between native tetracycline and combinatorial. The combinatorial tetracycline band is not separated either by two-dimensional TLC or by HPLC (not shown).

### Example 3. Obtaining a Supramolecular Combinatorial Mixture of Gentamicin (Aminoglycoside) (CMG)

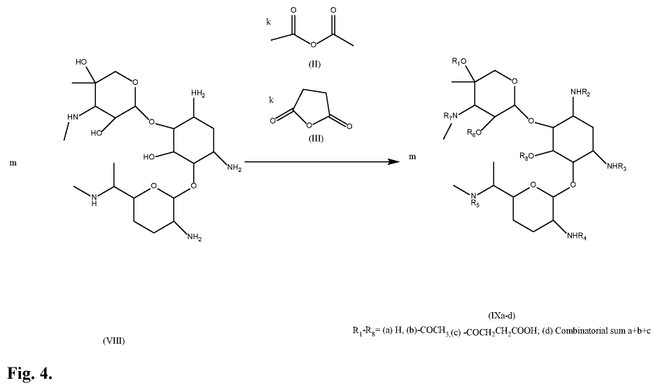

656 μM gentamicin base (VIII) (CAS N 1403-66-3, Mr=477.603 g/mol, n=8) (VIII) is dissolved in 10 ml of dioxane, 1750 μM of succinic anhydride (III) and 1750 μM of acetic anhydride are added (II), the solution is stirred and heated under reflux for 5-50 minutes. The solution was poured into ampoules and lyophilized to remove solvent and acetic acid. The combinatorial mixture (IXa-d) is used to obtain pharmaceutical compositions, study the structure, determine the biological activity (CMG). FIG. 4 shows a synthesis scheme for combinatorial derivatives of gentamicin.

In this reaction, instead of gentamicin, streptomycin, amikacin, or any other representative of aminoglycoside antibiotics with hydroxyl and amino groups available for modification, as well as any other antibiotic with two or more groups available for modification, can be used: aminoglycoside antibiotics, polyene antibiotics, tetracycline, macrolide antibiotics, lincosamine, gramicidin, glycopeptide antibiotics. Instead of modifiers of carboxylic acid anhydrides, halides of carboxylic and polycarboxylic acids, such as succinic, maleic, fumaric, lactic, propionic, other halogen derivatives, such as chloromethane, bromoethane, chloropropane, cyclic alkylating compounds, such as oxirane, and propoxy, can be used.

FIG. 4.

One source molecule of gentamicin (I) contains 8 hydroxyl and methyl amino groups available for modification (n=8).

Calculations of the number of moles of modifiers are carried out according to the combinatorics formulas:

(1)k=n(d+1)ⁿ⁻¹;(2)m=(d+1)ⁿ−1,

wherein

m=a number of different derivatives of molecules in the combinatorial mixture and the number of moles of tetracycline for the reaction;

n=a number of hydroxyl and amino groups available for modification in the structure of gentamicin (n=8); k is the number of moles of each modifier,

d=a number of modifiers=2.

Thus, having only one initial gentamicin molecule and two modifiers after combinatorial synthesis, we obtain 6560 combinatorial derivatives with different degrees of substitution, different positions of substituents and different permutations of the modifier residues, not just as a mixture, but as a difficult to separate supramolecular mixture.

Due to the presence in both derivatives of both substituted and non-substituted hydroxyl and methylamino groups, supramolecular structures are formed through both hydrogen and ionic bonds. Modifiers—succinic anhydride or acetic anhydride can be entered both simultaneously and sequentially—or first introduce succinic anhydride, warm the mixture under reflux, and then add acetic anhydride and re-warm the mixture. Similarly, in this reaction, maleic anhydride, aconitic anhydride, glutaric, phthalic anhydride and acetic anhydride, formic acid ethyl ester, monochloroacetic acid, propiolactone, ethylene oxide and other low molecular weight molecules, can be used as one of the modifiers instead of succinic anhydride.)

NMR C13: CH: 107.9; 107.1; 87.1; CH2: 63.8; C: 70.2; CH: 85.0; CH: 90.0; CH 64.4; CH 74.4; CH 65.0; CH 53.4; CH 55.7; CH 49.3; CH2 22.4; 34.8; 23.9; C: 170.2; 174.7; 173.8; 172.3; 173.0; CH: 60.2; CH3: 31.6; 34.0; 15.8; CH2: 29.8; 29.1; 30.2; 29.4; CH3: 21.1; 17.5;

C13 NMR data of the combinatorial derivative confirm the presence of both ethyl groups of succinic acid residues in its structure, and acetyl residues—reaction products with acetic anhydride.

For the HPLC, a Milichrom A-02 microcolumn chromatograph in a gradient of acetonitrile (5-100%)/0.1 M perchloric acid+0.5 M lithium perchlorate was used. The combinatorial derivative in the chromatogram gave one clear broadened peak and was not separated into components, although the retention time differed from both the original polymyxin and its completely substituted derivatives. This testified to the fact that complex supramolecular structures were formed between different combinatorial derivatives (in our case there were 6560 of them), which were not separated chromatographically. This combinatorial derivative (PMM) also behaves similarly when separated in a thin layer (acetonitrile: water, UV detection after treatment with 10% sulfuric acid solution) and gives only one band that does not coincide with any of the obtained derivatives.

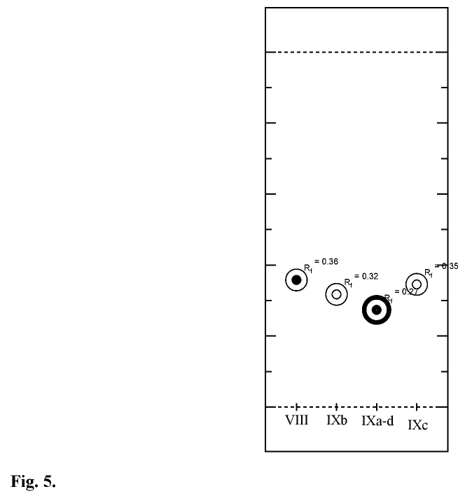

FIG. 5.

As can be seen from FIG. 5, TLC, the Combinatorial mixture (lane IXa-d) is less mobile and has Rf=0.27, while the initial unmodified gentamicin (VIII) is the lightest and Rf=0.36. Fully acylated gentamicin (IXb) and succinyl gentamicin (IXc) are intermediate between native gentamicin and combinatorial. The combinatorial gentamicin band is not separated either by two-dimensional TLC or by HPLC (not shown).

### Example 4. Determination of the Antimicrobial Activity of Patented Agents In Vitro

The objects of the study were 14 combinatorial derivatives of antibiotics: polymyxin (IV), tetracycline (VII), gentamicin (IX), streptomycin (X), lincomycin (XI), kanamycin (XII), erythromycin (XIII), midecamycin (XIV), amphotericin B (XV), vancomycin (XVI), nystatin (XVII), amikacin (XVIII), tobramycin (XIX), spiromycin (XX). The antimicrobial activity of the compounds was studied in a collection of test strains of microorganisms obtained from the Institute of Microorganisms Museum and living culture museums of various laboratories of the IMI NAMS State University (Kharkov). The collection included the following multiresistant strains: bacteria-Staphylococcus aureus (Staphylococcus aureus), E. coli (Escherichia coli), Shigella flexneri (dysentery bacillus), B. antracoides (anthracoid), Proteus vulgaris (vulgar protea), seudoonas aureinosa (stick) of mushrooms—Candida spp. (yeast-like fungi of the genus Candida), Microsporium Ian. (causative agent of microsporia), Trich. mentagrophytes (causative agent of trichophytosis), Aspergillus niger (aspergillus).

For the cultivation of bacteria, Hottinger broth (pH 7.2-7.4) was used, and for fungi, Saburo medium (pH 6.0-6.8). Antimicrobial and fungistatic activity was evaluated by the minimum inhibitory concentration (MIC)—the smallest amount of a substance that completely inhibited the growth of bacteria or fungi after cultivation. IPC was determined by the conventional method of serial dilutions with a coefficient of 2 in a liquid nutrient medium. For this purpose, the initial dilution of the test compound with a concentration of 50 μg/ml of culture medium (Hottinger broth) was prepared.

Subsequently, a sequential double dilution was carried out, as a result of which 25 ml were contained in 1 ml of culture medium; 12.5; 6.25; 3.12 μg/ml, etc.

The reference standard was nystatin and ethacridine lactate. This combinatorial mixture of antibiotic derivatives behaves like a quasi-fluid system—it adapts to the individual conditions of the body, preventing the emergence of resistance in bacteria. The results of studies of the antimicrobial and antifungal activity of derivatives of tannins are presented in table. 1.

As can be seen from the table. 1, the maximum antimicrobial activity against resistant strains of microorganisms was shown by all combinatorial derivatives of antibiotics, in contrast to their unmodified derivatives, which initially did not have antimicrobial activity against these strains.

Compounds (XV) and (XVII) belonging to the groups of derivatives of polyene antifungal agents showed antifungal activity at the level of the initial derivatives (31.25 μg/ml), while the initial unmodified amphotericin and nystatin did not exert antifungal activity on these resistant strains. Derivatives (IV), (XVIII), (XIX) (XX) had lower antifungal activity, although initially these antibiotics did not have antifungal activity at all, especially with respect to multiresistant strains. The maximum activity against almost all the studied microorganisms at a dose of 3.12 μg/ml was shown by the XII derivative or the supramolecular combinatorial kanamycin derivative, while the initial derivative did not have activity on these resistant strains at all.

TABLE 1
Antibacterial and fungistatic activity of supramolecular combinatorial derivatives of
antibiotics based on MIC, μg/ml

| | Strains of microorganisms * | | | | | | | | | |
|---|---|---|---|---|---|---|---|---|---|---|
| | S. | E. | S. | B. | P. | P. | C. | M. | T. | A. |
| | aureus | coli | flexneri | antracoides | aeruginosa | vulgaris | albicans | anosum | mentagraphytes | niger |
| Connection | IMI | IMI | IMI | IMI | IMI | IMI | IMI | IMI | IMI | IMI |
| number | res3 | res3 | res3 | res3 | res3 | res3 | res3 | res3 | res3 | res3 |
| IV | 3,12 | 3,12 | 6,25 | 6,25 | 250 | 250 | — | 250 | 250 | 250 |
| VII | 3,12 | 3,12 | 3,12 | 6,25 | 6,25 | — | — | — | — | — |
| IX | 3,12 | 3,12 | 3,12 | 6,25 | 6,25 | — | — | 250 | — | — |
| X | 6,25 | 6,25 | 6,25 | 6,25 | 12,5 | 250 | — | — | — | — |
| XI | 6,25 | 6,25 | 6,25 | 6,25 | 12,5 | 250 | — | — | — | — |
| XII | 3,12 | 3,12 | 3,12 | 3,12 | 3,12 | 3,12 | — | — | — | — |
| XIII | 6,25 | 6,25 | 6,25 | 6,25 | 3,12 | 6,25 | — | — | — | — |
| XIV | 6,25 | 6,25 | 6,25 | 3,12 | 6,25 | 6,25 | — | — | — | — |
| XV | — | — | — | — | — | — | 31,25 | 31,25 | 31,25 | 31,25 |
| XVI | 3,12 | 3,12 | 3,12 | 250 | 12,5 | 3,12 | — | — | — | — |
| XVII | — | — | — | — | — | — | 31,25 | 31,25 | 31,25 | 31,25 |
| XVIII | 6,25 | 6,25 | 6,25 | 6,25 | 12,5 | 6,25 | 31,25 | 250 | 250 | 250 |
| XIX | 6,25 | 6,25 | 6,25 | 6,25 | 3,12 | 6,25 | 31,25 | 250 | 250 | 250 |
| XX | 3,12 | 3,12 | 3,12 | 3,12 | 3,12 | 3,12 | 250 | 250 | 250 | 250 |
| [symbol] | 31,2 | 125 | — | — | — | — | 62,5 | 62,5 | 16,2 | 62,5 |
| [symbol] | | | | | | | | | | |

Notes:
— does not have activity in a dose of up to 500 mcg/ml;
* the initial unmodified derivatives of antibiotics did not affect the growth of these strains even at doses higher than 500 μg/ml.

Therefore, combinatorial supramolecular derivatives of antibiotics have potent antimicrobial and antifungal activity against multiresistant strains of microorganisms and fungi, whereas the initial unmodified antibiotics did not have such activity at all.

### Example 5. The Effectiveness of SCAA Against the Resistant Strain of Escherichia coli IMI2001 in the Model of Neutropenic Peritonitis/Sepsis in Mice and the Assessment of the Average Effective Dose (ED50)

The objective of this study was to study the dose response relationship after intravenous (iv) administration of a single dose of SKPA (CBT) in the range of 0.1612 mg/kg. The effect was investigated against resistant E. coli IMI2001 in a neutropenic peritonitis model. Administration of meropenem at a dose of 40 mg/kg was included as a positive control group. The number of colonies in the blood and peritoneal fluid was determined 5 hours after administration. The mouse peritonitis/sepsis model is a well-known model for antimicrobial activity studies as described by N. FrimodtMoller and J. D.

Knudsen in Handbook of Animal Models of Infection (1999), ed. by O. Zak & M. A. Sande, Academic Press, San Diego, US.

female outbred NMRI mice, 2530 grams (Harlan Scandinavia).

Escherichia coli IMI2001 from IMINAMN, Kharkov, Ukraine. Clinical isolate from a human wound from 2003 with multidrug resistance (to ampicillin, ceftazidime, aztreonam, gentamicin, ciprofloxacin).

ASCA in Ringer's acetate, pH 6, 1.2 mg/ml, 6.0 ml. The solution was stored at 4° C. until use. The analyzes of the compositions used for administration were performed at the end of the phase of the study conducted on live animals, and the following results were obtained in these analyzes.

TABLE 2
The ratio of concentrations in the experiment

| Estimated Concentration | | Measured concentration | |
|---|---|---|---|
| 1.2 | mg/ml | 1.16 | mg/ml |
| 0.6 | mg/ml | 0.53 | mg/ml |
| 0.3 | mg/ml | 0.28 | mg/ml |
| 0.15 | mg/ml | 0.12 | mg/ml |
| 0.075 | mg/ml | 0.047 | mg/ml |
| 0.03 | mg/ml | 0.040 | mg/ml |
| 0.016 | mg/ml | 0.002 | mg/ml |

Filler (Ringer's acetate, pH 6). The solution was stored at 4° C. until use.
Meronem (AstraZeneca, 500 mg infusion substance, meropenem). Sterile water.
Sterile 0.9% saline.
Cyclophosphamide, Apodan (APharma, 1 g). Agar plates and 5% horse blood.
Cups with agar, bromothymol blue and lactose.

Laboratory vivarium and mouse maintenance. Temperature and humidity in the vivarium were recorded daily. The temperature was 21° C.+/2° C. and could be controlled by heating and cooling. Humidity was 55+/10%. The change of air occurred approximately 1020 times per hour, and the period of light/darkness was in the 12 hour interval 06: 0018: 00/18: 00-06: 00. Mice had free access to drinking water for pets and food (2016, Harlan). Mice were kept in type 3 macrolon cells, 3 mice per cell. Tappen Aspen Wood was used as a litter. In addition, animals were given Sizzlenest paper strips as nest material. Mice were labeled with tails to distinguish between mice in the cage. Mice were weighed one day prior to administration.

Preparation of ASCA solutions

A solution with a concentration of 1.2 mg/ml was further diluted in PBS vehicle as follows.

TABLE 3
The ratio of the concentration of the substance and the filler

| | |
|---|---|
| 0.6 mg/ml~7.5 mg/kg: | 1.5 ml 1.2 mg/ml ASCA + 1.5 ml filler |
| 0.3 mg/ml~5.0 mg/kg: | 1.5 ml 0.6 mg/ml ASCA + 1.5 ml filler |
| 0.15 mg/ml~2.5 mg/kg: | 1.5 ml 0.3 mg/ml ASCA + 1.5 ml filler |
| 0.075 mg/ml~1.25 mg/kg: | 1.5 ml 0.15 mg/ml ASCA + 1.5 ml filler |
| 0.03 mg/ml~0.63 mg/kg: | 1.5 ml 0.075 mg/ml ASCA + 2.25 ml filler |
| 0.016 mg/ml~0.16 mg/kg: | 1.5 ml 0.03 mg/ml ASCA + 1.5 ml filler |

Preparation of a solution of meropenem. Administration of meropenem at a dose of 40 mg/kg was included as a positive control group. A total of 500 mg of meropenem (one ampoule) was dissolved in 10 ml of water at a concentration of about 50 mg/ml. This stock solution was further diluted to 4 mg/ml (0.4 ml, 50 mg/ml+4.6 ml saline).

Preparation of cyclophosphamide. A total of 1 g of cyclophosphamide (one Apodan 1 g ampoule) was dissolved in 50 ml of water, approximately 20 mg/ml, for each day of its use. This stock solution was further diluted to 11 mg/ml (16.5 ml, 20 mg/ml+13.5 ml of physiological saline) for use on day 4 or to 5 mg/kg (8.25 ml 20 mg/ml+21, 75 ml of physiological saline) for use on 1 day.

The introduction of cyclophosphamide to mice. Neutropenia was induced in mice by injection of 0.5 ml of cyclophosphamide solution intraperitoneally 4 days (200 mg/kg) and 1 day (100 mg/kg) before infection.

Infection of mice. Fresh E. coli IMI2001 colonies obtained overnight in an agar plate and 5% horse blood were suspended and diluted in sterile saline to approximately 2×10 6 CFU/ml. One hour before the start of administration (time point 1 h), mice were intraperitoneally infected with 0.5 ml of a suspension of E. coli in the lateral lower quadrant of the abdomen. Approximately 0.51 hours after the administration, 45 μl of neurofen (20 mg ibuprofen per ml, corresponding to 30 mg/kg) was orally administered to the mice as a painkiller.

The introduction of drugs to mice. Mice were given a single intravenous administration of CBT, meropenem, or excipient into the lateral tail vein for approximately 30 seconds in a volume of 10 ml/kg at time 0 h (see Table 1). The dose determination was based on an average body weight of 30 g. Mice with a body weight of 2832 g were injected with 0.30 ml of solution. Mice weighing 2728 g were injected with 0.25 ml of the solution and mice weighing 32.136 g were injected with 0.35 ml of the solution.

TABLE 4
Scheme of introduction and collection of samples in the model of
peritonitis in mice

| Infection, | Intravenous | Sampling and No | |
|---|---|---|---|
| intraperitoneally, 1 h | administration, 0 h | 0 h | 5 h |
| 0.5 ml E. coli IMI2001 | Filler, Ringer's acetate | | 1-2-3 |
| 1 × 10⁶ CFU/ml | CBT, 0.16 mg/kg | | 4-5-6 |
| | CBT, 0.30 mg/kg | | 7-8-9 |
| | CBT, 0.75 mg/kg | | 10-11-12 |
| | CBT, 1.5 mg/kg | | 13-14-15 |
| | CBT, 3.0 mg/kg | | 16-17-18 |
| | CBT, 6.0 mg / kg | | 19-20-21 |
| | CBT, 12 mg/kg | | 22-23-24 |
| | Meropenem, 40 mg/kg | | 25-26-27 |
| | Without injection | 28-29-30 | |

T indicates time relative to administration. The numbers in the columns of the sampling represent the identification numbers of mice. Clinical scoring of mice. Mice were observed during the study and assigned them scores from 0 to 5 depending on their behavior and clinical signs.
Score 0: healthy.
Score 1: minimal clinical signs of infection and inflammation, such as observing minimal signs of an upset or change in activity.
Score 2: distinct signs of infection, such as social self-isolation, decreased curiosity, altered body position, piloerection, or changes in the pattern of movement.
Score 3: pronounced signs of infection, such as stiffness, decreased curiosity, altered body position, piloerection, pain, or changes in the nature of movements.
Score 4: severe pain, and the mouse was immediately euthanized to minimize the suffering of the animal.
Score 5: mouse death.
   Fence samples.

The number of colonies was determined in blood and peritoneal fluid for 0 and 5 hours. Mice were anesthetized with CO2+02 and blood was taken from an axillary incision into Eppendorf tubes coated with ethylene diamine tetraacetic acid (EDTA). Mice were sacrificed immediately after blood sampling, and a total of 2 ml of sterile physiological saline was administered intraperitoneally and a gentle massage of the abdomen was performed until it was opened and the fluid samples were pipetted. Then, each sample was diluted 10 times in physiological saline and drops of 20 μl were applied to blue agar plates. All agar plates were incubated for 1822 hours at 35° C. in air.

Results. The number of colonies was determined at the beginning of administration and 5 hours after administration. The CFU count and clinical indicators of the mice are shown in Table 3. Before the calculations, a log 10 transformation of the CFU count was performed.

CFU/ml in the infectious material was determined to be 6.29 log 10. At the beginning of administration, the average log 10 CFU/ml was 5.76 in the peritoneal fluid and 5.13 in the blood, and the CFU levels were kept at a similar level in the filler group (5.72 and 4.65 log 10 CFU/ml in the peritoneal fluid and blood, respectively) 5 hours after administration. Slightly reduced CFU levels were observed in the blood and peritoneal fluid after administration of CPR at a dose of 0.163.0 mg/kg. The introduction of CPR at a dose of 6 and 12 mg/kg led to a significant decrease in the levels of CFU (p<0.001) compared with the introduction of the filler, both in the peritoneal fluid and in the blood (table 3). The introduction of meropenem at a dose of 40 mg/kg also led to a significant decrease compared with mice that were injected with excipient, both in the blood (p<0.05) and in the peritoneal fluid (p<0.01).

Dose response curves (data not shown) were calculated in GraphPad Prism using a sigmoid dose response curve (variable angle). The ED50 values determined from these curves were 2.11±1.01 mg/kg in the peritoneal fluid and 2.12±0.33 mg/kg in the blood. The maximum effect of CBT, Emax, was determined as the difference in log CFU in the absence of response and at the maximum response. The lack of response was characterized as the number of colonies at a level determined in mice that were injected with vehicle. Emax, calculated as the difference between the “Top plateau” and the “Bottom plateau” in GraphPad Prism using a sigmoid dose response curve, was 4.72 log 10 CFU for peritoneal fluid and 3.15 log 10 CFU for blood. In addition, 1, 2, and 3 log kills were estimated using GraphPad Prism, defined as the dose required to reduce the bacterial load by 1, 2, or 3 log compared to the start of treatment. 1, 2 and 3 log kill for CBT were 1.11 mg/kg, 2.95 mg/kg and 4.73 mg/kg, respectively, in peritoneal fluid and 0.25 mg/kg, 2.75 mg/kg and 3.78 mg/kg, respectively, in the blood. In all administration groups, zero or low clinical scores were observed (Table 3).

Discussion and conclusion. The objective of this study was to study the dose response relationship after intravenous (iv) administration of a single dose of CBT in the range of 0.1612 mg/kg. The effect was investigated against E. coli IMI2001 in a neutropenic peritonitis/sepsis model. It was determined that ED50 values for CBT were 2.11±1.01 mg/kg in peritoneal fluid and 2.12±0.33 mg/kg in blood. An estimated 1 log kill was 1.11 mg/kg in peritoneal fluid and 0.25 mg/kg in blood. An estimated 2 log destruction was 2.95 mg/kg in peritoneal fluid and 2.76 mg/kg in blood.

An estimated 3 log kill was 4.73 m/k in peritoneal fluid and 3.78 m/k in blood.

TABLE 5
The effectiveness of CBT against E. coli IMI2001 calculated in
GraphPad Prism

| CBT | Peritoneal fluid | | Blood | |
|---|---|---|---|---|
| The greatest | 0.325 | CFU/ml | −0.985 | CFU/ml |
| The greatest | −4.486 | CFU/ml | −4.138 | CFU/ml |
| Emax | 4.811 | CFU/ml | 3.153 | CFU/ml |
| ED50 | 2.11 | mg/kg | 2.12 | mg/kg |
| R2 | 0.7524 | | 0.6889 | |
| 1 log destruction | 1.11 | mg/kg | 0.25 | mg/kg |
| 2 log destruction | 2.95 | mg/kg | 2.76 | mg/kg |
| 3 log destruction | 4.73 | mg/kg | 3.78 | mg/kg |

TABLE 6
The amount of ASCA in the blood and peritoneal fluid of mice with neutropenia,
which was administered a single dose of ASCA, meropenem or excipient

| Injection | | | Clinical Score | | log₁₀ CFU | | | |
|---|---|---|---|---|---|---|---|---|
| T = 0 h | No mice | Time | T = 0 h | T = 5 h | PF | Average in PF | Blood | Blood average |
| Filler | 1 | T = 5 | 1 | 1 | 5,74 | | 5,05 | |
| | 2 | T = 5 | 1 | 0 | 5,54 | 5,72 | 4,78 | 4,65 |
| | 3 | T = 5 | 1 | 1 | 5,88 | | 4,H | |
| CBT | 4 | T = 5 | 1 | 1 | 5,16 | | 4,27 | |
| 0,16 mg/ | 5 | T = 5 | 1 | 1 | 4,78 | 5,31 | 4,19 | 4,54 |
| kg | 6 | T = 5 | 1 | 0 | 5,98 | | 5,16 | |
| CBT | 7 | T = 5 | 1 | 0 | 2,76 | | 1,40 | |
| 0,30 mg/ | 8 | T = 5 | 1 | 1 | 5,74 | 4,26 | 4,63 | 2,88 |
| kg | 9 | T = 5 | 1 | 0 | 4,27 | | 2,60 | |
| CBT | 10 | T = 5 | 1 | 0 | 5,74 | | 5,07 | |
| 0,75 mg/ | 11 | T = 5 | 1 | 1 | 4,95 | 5,16 | 4,30 | 4,46 |
| kg | 12 | T = 5 | 1 | 1 | 4,78 | | 4,00 | |
| CBT | 13 | T = 5 | 1 | 0 | 3,33 | | 3,51 | |
| 1,5 mg/ | 14 | T = 5 | 1 | 1 | 4,72 | 4,41 | 3,92 | 3,99 |
| kg | 15 | T = 5 | 1 | 0 | 5,18 | | 4,54 | |
| CBT | 16 | T = 5 | 1 | 1 | 4,74 | | 3,86 | |
| 3,0 mg/ | 17 | T = 5 | 1 | 1 | 4,74 | 3,91 | 3,57 | 2,81 |
| kg | 18 | T = 5 | 1 | 0 | 2,24 | | 1,00 | |
| CBT | 19 | T = 5 | 1 | 1 | 2,18 | 2,12 | 1,00 | 1,00 |
| 6,0 mg/ | 20 | T = 5 | 1 | 1 | 2,18 | *** | 1,00 | *** |
| kg | 21 | T = 5 | 1 | 1 | 2,00 | | 1,00 | |
| CBT | 22 | T = 5 | 1 | 1 | 1,00 | 1,36 | 1,00 | 1,00 |
| 12 mg/ | 23 | T = 5 | 1 | 0 | 1,69 | *** | 1,00 | *** |
| kg | 24 | T = 5 | 1 | 0 | 1,40 | | 1,00 | |
| Merope | 25 | T = 5 | 1 | 1 | 3,92 | 2,64 | 2,48 | 2,38 |
| nem 40 | 26 | T = 5 | 1 | 1 | 1,70 | ** | 1,70 | * |
| mg/kg | 27 | T = 5 | 1 | 0 | 2,30 | | 2,95 | |
| No | 28 | T = 0 | 1 | | 5,84 | | 5,08 | |
| | 29 | T = 0 | 1 | | 5,78 | 5,76 | 4,98 | 5,13 |
| | 30 | T = 0 | 1 | | 5,65 | | 5,34 | |

PF peritoneal fluid
Asterisks indicate significant differences from the filler group (analysis of variance, multiple comparison).
* corresponds to p < 0.05;
** corresponds to p < 0.01;
*** corresponds to p < 0.001.

The detection limit is 1.4 log 10 CFU/ml. Samples without detected bacteria are presented as 1.0 log 10 CFU/ml.

### Example 6. The Model of Peritonitis/Sepsis: The Effect of CAT at a Dose of 7.5 mg/kg Over Time Against Escherichia coli IMI2001 in NMRI Mice with Neutropenia

The objective of this study was to investigate the effectiveness of CPR in vivo after intravenous (iv) administration of a single dose of 7.5 mg/kg. The effect was tested against Escherichia coli IMI2001 in a peritonitis model in neutropenia NMRI mice to avoid the use of mucin, which is commonly used in a mouse peritonitis model. Neutropenia was induced in mice by injection of cyclophosphamide. Administration of meropenem at a dose of 40 mg/kg was included as a positive control group and vehicle administration was included as a negative control group. The number of colonies in peritoneal fluid and blood was determined 2 and 5 hours after administration.

30 female outbred mice NMRI, 2832 grams (Kiev).
Escherichia coli IMI2001 from IMINAMN, Ukraine, Kharkov. Clinical isolate from a human wound from 2013 with multidrug resistance (to ampicillin, ceftazidime, aztreonam, gentamicin, ciprofloxacin).
PPC in Ringer's acetate, pH 6, 1.2 ml, 0.75 mg/ml. Assays of the administered compositions performed after the study showed a concentration of approximately 0.78 mg/ml.
Filler (Ringer's acetate, pH 6), 3 ml.
Meronem (AstraZeneca, 500 mg infusion substance, Apodan meropenem (APharma, 1 g cyclophosphamide).
Sterile water.
Sterile 0.9% saline.
Agar plates and 5% horse blood.
Cups with agar, bromothymol blue and lactose.

Laboratory vivarium and mouse maintenance. Temperature and humidity in the vivarium were recorded daily. The temperature was 21° C.+/2° C. and could be controlled by heating and cooling. Humidity was 55+/10%. The change of air took place approximately 1020 times per hour, and the period of light/darkness was in the 12 hour interval 06: 0018: 00/18: 0006: 00. Mice had free access to drinking water for pets and food (2016, Harlan). Mice were kept in type 3 macrolon cells, 3 mice per cell. Tappen Aspen Wood was used as a litter. In addition, animals were given Sizzlenest paper strips as nest material.

Mice were labeled with tails to distinguish between mice in the cage.

CAT solution. A solution of CPP with a concentration of 0.75 mg/ml was stored at +4° C. for up to one hour before injection, then at room temperature.

Preparation of a solution of meropenem. A total of 500 mg of meropenem (one ampoule) was dissolved in 10 ml of water, approximately 50 mg/ml, on the day of its use. This stock solution was further diluted to 4 mg/ml (0.4 ml, 50 mg/ml+4.6 ml saline).

Preparation of cyclophosphamide. A total of 1 g of cyclophosphamide (one Apodan ampoule) was dissolved in 50 ml of water, approximately 20 mg/ml, for each day of its use. This stock solution was further diluted to 11 mg/ml (16.5 ml, 20 mg/ml+13.5 ml of physiological saline) for use on day 4 or to 5 mg/kg (8.25 ml 20 mg/ml+21, 75 ml of physiological saline) for use on 1 day.

The introduction of cyclophosphamide to mice. Neutropenia was induced in mice by injection of 0.5 ml of cyclophosphamide solution intraperitoneally 4 days (200 mg/kg) and 1 day (100 mg/kg) before infection.

Infection of mice. Fresh E. coli AID #172 colonies obtained overnight in an agar plate and 5% horse blood were suspended and diluted in sterile saline to approximately 2×10 6 CFU/ml. One hour before the start of administration (time point 1 h), mice were intraperitoneally infected with 0.5 ml of a suspension of E. coli in the lateral lower quadrant of the abdomen. 2.5 h after administration with significant clinical signs of infection, mice were orally administered 45 μl of neurofen (20 mg ibuprofen per ml, which corresponded to 30 mg/kg) as a painkiller.

Score mice. At each sampling in mice, a clinical assessment of the clinical signs of infection was performed.
Score 0: healthy.
Score 1: minimal clinical signs of infection and inflammation, for example, observation of minimal signs of an upset or activity change
Score 2: distinct signs of infection, such as social self-isolation, decreased curiosity, altered body position, piloerection, or changes in the pattern of movement.
Score 3: pronounced signs of infection, such as stiffness, decreased curiosity, altered body position, piloerection, pain, or changes in the nature of movements.
Score 4: severe pain, and the mouse was immediately euthanized to minimize the suffering of the animal.
Score 5: mouse death.

The introduction of drugs to mice. The mice were given a single intravenous administration of CPR, meropenem, or excipient into the lateral tail vein for approximately 30 seconds at a time point of 0 h (see Table 6). The dose determination was based on an average body weight of 30 g. Mice with a body weight of 2832 g were injected with 0.30 ml of solution. Mice weighing 2728 g were injected with 0.25 ml of the solution and mice weighing 32.136 g were injected with 0.35 ml of the solution. Mice 17 accidentally injected 0.35 ml, despite the fact that its body weight was 29.5 g. Apparently, this did not affect the results, since the CFU levels in this mouse were very similar to two other mice in this group.

TABLE 7
The scheme of introduction and sampling in the model of peritonitis in mice

| | | Fence samples | Fence samples | Fence samples |
|---|---|---|---|---|
| InfectionT = −1 h | Injection T = 0 h | T = 0 h | T = 2 h | T = 5 h |
| 0.5 ml | PPC-Gearbox | | | 4, 5, 6 |
| E. coli IMI2001 = 10⁶ | Meropenem | | | 7, 8, 9 |
| CFU/ml | Filler (Ringer's acetate) | | | 10, 11, 12 |
| | PPC | | 16, 17, 18 | |
| | Meropenem | | 19, 20, 21 | |
| | Filler (Ringer's acetate) | | 22, 23, 24 | |
| | Non | | 25, 26, 27 | |
| | Non | 28, 29, 30 | | |

T indicates time relative to administration. The numbers in the sampling columns are the mouse
identification numbers.

Fence samples. The number of colonies was determined in blood and peritoneal fluid at 0, 2 and 5 hours after administration according to Table 6.

Mice were anesthetized with CO2+O2 and blood sampling was performed from a section in the axillary region. The mice were sacrificed by cervical dislocation and a total of 2 ml of sterile physiological saline was injected intraperitoneally and a gentle massage of the abdomen was performed, then it was opened and a sample of the fluid was pipetted. Each sample was diluted 10 times in saline and drops of 20 μl were applied to plates with blood agar. All agar plates were incubated for 1822 hours at 35° C. in air.

Results. The number of colonies and clinical indicators of mice are shown in Table 2. Before the calculations, a log 10 transformation of the number of CFU was performed to obtain a normal distribution. CFU/ml in the infectious material was determined to be 6.50 log 10. At the beginning of administration, the average log 10 CFU/ml was 3.57 in the peritoneal fluid and 3.54 in the blood, and the level of CFU increased to 5.43 and 4.58 in the peritoneal fluid and blood, respectively, after 2 hours in animals that were injected filler, and up to 5.72 and 4.74 in the peritoneal fluid and blood, respectively, after 5 hours in mice that were injected with the filler, which was to be expected. 2 hours after the administration of CPT, significantly reduced CFU levels were observed, both in the blood and in the peritoneal fluid, compared with the administration of an excipient (p<0.001). An additional decrease in CFU levels, both in the blood and in the peritoneal fluid, was observed through 5 hours after administration of CPR (p<0.001 compared with the control vehicle).

CFU levels were more than 3 log₁₀ CFU/ml lower than after vehicle administration. Administration of meropenem also led to a significant (p<0.01) decrease in CFU levels compared to vehicle administration in peritoneal fluid 2 and 5 hours after administration. but in the blood only 5 hours after administration. The absence of a significant decrease in blood 2 hours after administration may reflect more pronounced variability in the filler group, rather than a weak effect of meropenem. Differences in CFU levels after administration of CPR or meropenem compared to vehicle administration were as follows.

TABLE 8
Differences in CFU levels after administration of ASCA or meropenem compared with
the injection of filler

| | | |
|---|---|---|
| ASCA, 7.5 mg/kg | 2 h: peritoneum −1.63 log CFU/ml | blood −2.50 log CFU/ml |
| Gearbox | 5 h: peritoneum −3.76 log CFU/ml | blood −3.74 log CFU/ml |
| Meropenem, 40 mg/kg | 2 h: peritoneum −1.51 log CFU/ml | blood −0.82 log CFU/ml |
| | 5 h: peritoneum −1.51 log CFU/ml | blood −1.64 log CFU/ml |

All mice had mild symptoms of infection or no symptoms of infection.

Discussion and conclusion. The objective of this study was to investigate the effectiveness of CPR after intravenous (iv) administration of a single dose of 7.5 mg/kg in a model of neutropenic peritonitis in NMRI mice. For CPR, a significant (p<0.001) decrease of more than 3 log 10 CFU/ml was observed compared with the administration of the excipient in the blood and peritoneal fluid 5 hours after administration. In addition, a significant decrease (p<0.001) was observed both in the blood and in the peritoneal fluid 2 hours after the administration of CPT. Meropenem showed a significant decrease compared with the filler group (p<0.01) both in the blood and in the peritoneal fluid after 5 hours, but 2 hours after administration only in the peritoneal fluid.

TABLE 9
The number of colonies of E. coli IMI2001 in mice that were administered a single dose
of CPR, filler or meropenem

| | | | | | | log₁₀ CFU | | | |
|---|---|---|---|---|---|---|---|---|---|
| (Injection) or | | Sampling | Score | | | | [symbol] | | [symbol] |
| (Delivery) | No | Time | T = 0 h | T = 2 h | T = 5 h | PF | [symbol] PF | Blood | [symbol] |
| PPC CPR | 4 | T = 5 | 0 | | 0 | 2,18 | 1 96 *** | 1,00 | 1,00 *** |
| 7,5 mg / kg | 5 | T = 5 | 0 | | 1 | 2,30 | | 1,00 | |
| | 6 | T = 5 | 0 | | 1 | 1,40 | | 1,00 | |
| Meropenem | 7 | T = 5 | 0 | | 0 | 4,38 | 4,21 ** | 3,20 | 3,10 ** |
| 40 mg / kg | 8 | T = 5 | 0 | | 0 | 4,00 | | 2,85 | |
| | 9 | T = 5 | 0 | | 0 | 4,26 | | 3,26 | |
| | 10 | T = 5 | 0 | | 0 | 5,39 | | 4,63 | |
| Filler | 11 | T = 5 | 0 | | 0 | 5,99 | 5,72 | 5,24 | 4,74 |
| | 12 | T = 5 | 0 | | 0 | 5,78 | | 4,36 | |
| PPC | 16 | T = 2 | 0 | 1 | | 4,24 | 3,79 ** | 2,04 | 2,08 ** |
| 7,5 mg / kg | 17 | T = 2 | 0 | 1 | | 3,60 | | 2,20 | |
| | 18 | T = 2 | 0 | 1 | | 3,54 | | 2,00 | |
| Meropenem | 19 | T = 2 | 0 | 1 | | 4,12 | 3,92 ** | 3,60 | 3,76 |
| 40 mg / kg | 20 | T = 2 | 0 | 1 | | 3,40 | | 3,57 | |
| | 21 | T = 2 | 0 | 1 | | 4,24 | | 4,H | |
| | 22 | T = 2 | 0 | 0 | | 4,89 | | 3,21 | |
| Filler | 23 | T = 2 | 0 | 1 | | 5,65 | 5,43 | 5,39 | 4,58 |
| | 24 | T = 2 | 0 | 0 | | 5,74 | | 5,15 | |
| | 25 | T = 2 | 0 | 0 | | 4,45 | | 4,39 | |
| No | 26 | T = 2 | 0 | 0 | | 5,42 | 5,08 | 4,57 | 4,33 |
| | 27 | T = 2 | 0 | 0 | | 5,38 | | 4,02 | |
| | 28 | T = 0 | 0 | | | 1,88 | | 1,00 | |
| No | 29 | T = 0 | 0 | | | 3,71 | 3,57 | 4,27 | 3,54 |
| | 30 | T = 0 | 0 | | | 5,13 | | 5,35 | |

PF peritoneal fluid. Used infectious material: 1.97 × 106 CFU/ml.
* Mice were injected with 0.35 ml instead of 0.30 ml of the test compound.
* p < 0.05;
** p < 0.01;
*** p < 0.001 compared to the filler group.

### Example 7. A Model of Hip Infection with Neutropenia: The Effectiveness of CPR Against Escherichia coli IMI2001H OueHKa ED50

Introduction The objective of this study was to study the dose response after intravenous (iv) administration of a single dose of CPR in the range of 0.1612 mg/kg. The effect was investigated against E. coli IMI2001 in a model of femoral infection with neutropenia. Administration of meropenem at a dose of 40 mg/kg was included as a positive control group. The number of colonies in the hips was determined 5 hours after administration. The hip infection model is a well-known model for studies of the antimicrobial effect and tissue penetration, as described by S. Gudmundsson & N. Erlensdottir: Handbook of Animal Models of Infection (1999), ed. by O. Zak & M. A. Sande, Academic Press, San Diego, US, and some other publications. See the review by D. Andes & C. Craig: Animal model pharmacokinetics and pharmacodynamics: a critical review. International Journal of Antimicrobial Agents, 19 (4): 261268.

Materials and methods. 40 female outbred mice NMRI, 2530 grams (Kiev, Ukraine). Escherichia coli IMI2001 from IMINAMN, Kharkov, Ukraine. Clinical isolate from a human wound from 20q3 with multidrug resistance (to ampicillin, ceftazidime, aztreonam, gentamicin, ciprofloxacin).

PPC in Ringer's acetate, pH 6, 1.2 mg/ml, 6.0 ml. The solution was stored at 4° C. until use. The analyzes of the compositions used for administration were performed at the end of the phase of the study conducted on live animals, and the following results were obtained in these analyzes.

TABLE 10
The concentration of the drugs used for experiment

| Estimated Concentration | | Measured concentration | |
|---|---|---|---|
| 1.2 | MΓ/ml | 1.4 | MΓ/ml |
| 0.6 | MΓ/ml | 0.57 | MΓ/ml |
| 0.3 | MΓ/ml | 0.28 | MΓ/ml |
| 0.15 | MΓ/ml | 0.15 | MΓ/ml |
| 0.075 | MΓ/ml | 0.063 | MΓ/ml |
| 0.03 | MΓ/ml | 0.02 | MΓ/ml |
| 0.016 | MΓ/ml | 0.014 | MΓ/ml |

Filler (Ringer's acetate, pH 6). The solution was stored at 4° C. until use.

Meronem (AstraZeneca, 500 mg infusion substance, meropenem). Lot number: 09466C. Expiration date: August 2013

Sterile water.

Sterile 0.9% saline.

Sendoxan (cyclophosphamide, Baxter, 1 g). Lot number: 0A671C. Shelf life:

January 2013

Agar plates and 5% horse blood.

Agar, bromothymol blue and lactose cups

Laboratory vivarium and mouse maintenance. Temperature and humidity in the vivarium were recorded daily. The temperature was 21° C.+/2° C. and could be controlled by heating and cooling. Humidity was 55+/10%. The change of air occurred approximately 1020 times per hour, and the period of light/darkness was in the 12 hour interval 06: 0018: 00/18: 00-06: 00. Mice had free access to drinking water for pets and food (2016, Harlan). Mice were kept in type 3 macrolon cells, 4 mice per cell. Tappen Aspen Wood was used as a litter. In addition, animals were given Sizzlenest paper strips as nest material. Mice were labeled with tails to distinguish between mice in the cage. Mice were weighed one day prior to administration.

Preparation of ASCA solutions. A solution with a concentration of 1.2 mg/ml was further diluted in PBS vehicle as follows.

TABLE 11
Dosage of drugs in different concentrations and forms.

| | |
|---|---|
| 0.6 MΓ/ml~7.5 mg/kg: | 1.5 ml 1.2 MΓ/ml PPC + 1.5 ml filler |
| 0.3 MΓ/ml~5.0 mg/kg: | 1.5 ml 0.6 MΓ/ml PPC + 1.5 ml filler |
| 0.15 MΓ/ml~2.5 mg/kg: | 1.5 ml 0.3 MΓ/ml PPC + 1.5 ml filler |
| 0.075 MΓ/ml~1.25 mg/kg: | 1.5 ml 0.15 MΓ/ml PPC + 1.5 ml filler |
| 0.03 MΓ/ml~0.63 mg/kg: | 1.5 ml 0.075 MΓ/ml PPC + 2.25 ml filler |
| 0.016 MΓ/ml~0.16 mg/kg: | 1.5 ml 0.03 MΓ/ml PPC + 1.5 ml filler |

Preparation of a solution of meropenem. Administration of meropenem at a dose of 40 mg/kg was included as a positive control group. A total of 500 mg of meropenem (one ampoule) was dissolved in 10 ml of water, approximately 50 mg/ml. This stock solution was further diluted to 4 mg/ml (0.4 ml, 50 mg/ml+4.6 ml saline).

Preparation of cyclophosphamide. A total of 1 g of cyclophosphamide (one Sendoxan ampoule of 1 g) was dissolved in 50 ml of water, approximately 20 mg/ml, for each day of its use. This stock solution was further diluted to 11 mg/ml (16.5 ml, 20 mg/ml+13.5 ml of physiological saline) for use on day 4 or to 5 mg/kg (8.25 ml 20 mg/ml+21, 75 ml of physiological saline) for use on 1 day.

The introduction of cyclophosphamide to mice. Neutropenia was induced in mice by injection of 0.5 ml of cyclophosphamide solution intraperitoneally 4 days (200 mg/kg) and 1 day (100 mg/kg) before infection.

Infection of mice. Fresh E. coli IMI2001 colonies obtained overnight in an agar plate and 5% horse blood were suspended and diluted in sterile saline to approximately 2×107 CFU/ml. One hour before the start of administration (time point 1 h), intramuscular infection of mice with 0.05 ml of a suspension of E. coli in the left hind paw was performed. Approximately 0.5 hours after the administration, 45 μl of neurofen (20 mg ibuprofen per ml, corresponding to 30 mg/kg) was orally administered to the mice as a painkiller.

The introduction of drugs to mice. Mice were given a single intravenous administration of ASCA, meropenem, or excipient into the lateral tail vein for approximately 30 seconds in a volume of 10 ml/kg at time 0 h (see Table 1). The dose determination was based on an average body weight of 30 g. Mice with a body weight of 2832 g were injected with 0.30 ml of solution. Mice weighing 2728 g were injected with 0.25 ml of the solution and mice weighing 32.136 g were injected with 0.35 ml of the solution.

TABLE 12
Scheme of administration and collection of samples in a model of
femoral infection in mice

| Infection, | | Fence samples, and NoNo | |
|---|---|---|---|
| intramuscularly, | Intravenous | mice | |
| l h | administration 0 h | 0 h | 5 h |
| 0,05 ml E. coli | Filler, aueTaT PHΓepa | | 1-2-3-4 |
| IMI20012 × 10⁷ | PPC, 0.16 mg/kg | | 5-6-7-8 |
| CFU/ml | PPC, 0.30 mg/kg | | 9-10-11-12 |
| | PPC, 0.75 mg/kg | | 13-14-15-16 |
| | PPC, 1.5 mg/kg | | 17-18-19-20 |
| | PPC, 3.0 mg/kg | | 21-22-23-24 |
| | PPC, 6.0 mg/kg | | 25-26-27-28 |
| | PPC, 12 mg/kg | | 29-30-31-32 |
| | Meropenem, 40 mg/kg | | 33-34-35-36 |
| | Without administration | 37-38-39-40 | |

T indicates time relative to administration, the numbers in the fence
samples columns are the mouse identification numbers.

Clinical scoring of mice. Mice were observed during the study and assigned them scores from 0 to 5 depending on their behavior and clinical signs.
Score 0: healthy.
Score 1: minimal clinical signs of infection and inflammation, for example, observing minimal signs of an upset or change in activity.
Score 2: distinct signs of infection, such as social self-isolation, decreased curiosity, altered body position, piloerection, or changes in the pattern of movement.
Score 3: pronounced signs of infection, such as stiffness, decreased curiosity, altered body position, piloerection, pain, or changes in the nature of movements.
Score 4: severe pain, and the mouse was immediately euthanized to minimize the suffering of the animal.
Score 5: mouse death.

### Sampling

The number of colonies was determined in the hips at 0 and 5 h. Mice were anesthetized with CO2+O2 and killed. Immediately after this, the skin was removed, the hind left paw was received and it was frozen at 70° C. After thawing, the hips were homogenized using Dispomix Drive. Then, each sample was diluted 10 times in physiological saline and drops of 20 μl were applied to blue agar plates. All agar plates were incubated for 1822 hours at 35° C. in air.

Results. The number of colonies was determined at the beginning of administration and 5 hours after administration. The number of CFU is shown in Table 3. Before the calculations, a log 10 transformation of the number of CFU was performed. CFU/ml in the infectious material was determined to be 7.35 log 10, which corresponds to 6.05 log 10 CFU/mouse. The observed high variability may be due to suboptimal infection of some mice, leading to too low CFU. For this reason, the smallest value in each group was excluded from the graphs and calculations (see Table 3). At the beginning of administration, the average log 10 CFU/ml was 4.93 and increased to 6.49 log 10 CFU/ml in the filler group 5 hours after administration. Slightly reduced CFU levels were observed after administration of CPR at a dose of 0.163.0 mg/kg.

After administration of ASCA at a dose of 6 mg/kg (p<0.05) and 12 mg/kg (p<0.01), a significant decrease in CFU levels was observed compared with the administration of excipient (Table 9). The administration of meropenem at a dose of 40 mg/kg resulted in a definite but insignificant decrease compared to the mice that were injected with the vehicle. Dose response curves (not shown) were calculated in GraphPad Prism using a sigmoid dose response curve (variable angle of inclination). The ED50 determined from these curves was 5.9 mg/kg. However, no lower plateau has been received, and therefore this value may be underestimated.

The maximum ASCA effect, Emax, was determined as the difference in log CFU in the absence of response and at the maximum response. The lack of response was characterized as the number of colonies at a level determined in mice that were injected with vehicle. Emax, calculated as the difference between the Upper Plateau and the Lower Plateau in GraphPad Prism using a sigmoid dose response curve, was 2.4 log 10 CFU/ml. In addition, 1 log kill, defined as the dose required to reduce the bacterial load by 1 log compared to the start of treatment, determined using GraphPad Prism, was 6.1 mg/kg. 2 and 3 log destruction were not received.

No mouse showed a clinical sign of infection at any time point.

TABLE 13
ASCA Effectiveness Against E. coli AID #
172 Calculated in GraphPad Prism

| | |
|---|---|
| The greatest | 1.1 Δlog10 CFU/ml |
| The greatest | −1.3 Δlog₁₀ CFU/ml |
| Emax | 2.4 Δlog10 CFU/ml |
| ED50 | 5.9 mg/kg |
| R2 | 0.46 |
| 1 log destruction | 6.1 mg/kg |

TABLE 14
E. coli IMI2001 mice with neutropenia in the thighs
of mice injected with a single dose of
PPC, Meropenema or filler

| | No | | log₁₀ | |
|---|---|---|---|---|
| Administration | the | Sampling | CFU, | The |
| T = 0 h | mouse | Time | hip | average |
| Filler | 1 | T = 5 | 5,16¤ | 6,49 |
| | 2 | T = 5 | 6,47 | |
| | 3 | T = 5 | 6,13 | |
| | 4 | T = 5 | 6,86 | |
| PPC | 5 | T = 5 | 3,18¤ | 5,16 |
| 0,16 mg/kg | 6 | T = 5 | 6,03 | |
| | 7 | T = 5 | 3,30 | |
| | 8 | T = 5 | 6,15 | |
| PPC | 9 | T = 5 | 2,00¤ | 5,09 |
| 0,30 mg/kg | 10 | T = 5 | 5,40 | |
| | 11 | T = 5 | 3,10 | |
| | 12 | T = 5 | 6,78 | |
| PPC | 13 | T = 5 | 2,9¤ | 6,33 |
| 0,75 mg/kg | 14 | T = 5 | 5,72 | |
| | 15 | T = 5 | 7,27 | |
| | 16 | T = 5 | 6,00 | |
| PPC | 17 | T = 5 | 2,56¤¤ | 5,62 |
| 1,5 mg/kg | 18 | T = 5 | 6,23 | |
| | 19 | T = 5 | 4,93 | |
| | 20 | T = 5 | 5,70 | |
| PPC 3,0 mg/kg | 21 | T = 5 | 5,30 | 6,07 |
| | 22 | T = 5 | 6,03 | |
| | 23 | T = 5 | 4,85¤ | |
| | 24 | T = 5 | 6,89 | |
| PPC 6,0 mg/kg | 25 | T = 5 | 2,75 | 4,10* |
| | 26 | T = 5 | 4,54 | |
| | 27 | T = 5 | l,48¤ | |
| | 28 | T = 5 | 5,01 | |
| PPC 12 mg/kg | 29 | T = 5 | 2,48¤ | 3,32** |
| | 30 | T = 5 | 3,27 | |
| | 31 | T = 5 | 3,19 | |
| | 32 | T = 5 | 3,51 | |
| Meropenem | 33 | T = 5 | 3,08¤ | 4,25 |
| 40 mg/kg | 34 | T = 5 | 3,81¤ | |
| | 35 | T = 5 | 4,99 | |
| | 36 | T = 5 | 3,94 | |
| Without | 37 | T = 0 | 4,98 | 4,93 |
| administration | 38 | T = 0 | 3,81¤ | |
| | 39 | T = 0 | 4,79 | |
| | 40 | T = 0 | 5,01 | |

¤This value was excluded from the calculations, since its salts are a drop-out value.
Asterisks indicate significant differences from the filler group (analysis of variance, multiple comparison).
*corresponds to p < 0.05;
** corresponds to p < 0.01.

The detection limit is 1,4 log₁₀ CFU/ml. The scope of the invention described and claimed herein should not be limited to the specific aspects disclosed herein, since these aspects are merely illustrative of certain aspects of the invention. Assume that any equivalent aspects are included in the scope of this invention. Indeed, various modifications of the invention, in addition to those shown and described herein, will be apparent to those skilled in the art from the foregoing description. Assume that such modifications are also included in the scope of the attached claims. In the event of a conflict, this description, including definitions, should be followed.

1. New derivatives of antibiotics: polymyxin, amikacin, gentamicin, tetracycline, lincosamine, gramicidin, nystatin, erythromycin, nystatin, which are obtained by the reaction of acylation or/and alkylation of the original antibiotic simultaneously with at least two different modifiers, wherein synthesized individual derivatives self-organize into a supramolecular structure, while the molar ratio of antibiotic to modifiers in the reaction of combinatorial synthesis is calculated by the formulas:
k=n(d+1)ⁿ⁻¹ (1)
m=(d+1)ⁿ−1 (2)
wherein:
n=a number of groups —OH and —NH₂ and —NHR— available for substitution in the antibiotic;
m=a number of moles of the antibiotic and the number of different derivatives after synthesis;
k=a number of moles of each of d modifiers in the combinatorial synthesis reaction to obtain the maximum number of different derivatives;
d=a number of modifiers in combinatorial reaction;
and this synthesized supramolecular structure used for creating pharmaceutical compositions.

2. The new derivatives of antibiotics according to claim 1, wherein the modifiers are acylating agents of the group of anhydrides of organic mono— and polycarboxylic acids

3. The new derivatives of antibiotics according to claim 1, wherein the modifiers are halides of carboxylic acids

4. The new derivatives of antibiotics according to claim 1, wherein the modifiers are alkylating agents halogen derivatives of hydrocarbons

5. The new derivatives of antibiotics according to claim 1, wherein the modifiers are an acylating and alkylating agents.
